(19) United States
(12) Patent Application Publication
(10) Pub. No.: US 2008/0281418 A1
(43) Pub. Date: Nov. 13, 2008

(54) BREAST IMPLANT ARTICLES OF MULTI-LAYERED SHEETS OF EXTRACELLULAR MATRIX OR BALLED STRIPS AND PIECES OF EXTRACELLULAR MATRIX

(75) Inventor: LEIGH H. FIRESTONE, Berkeley, CA (US)

Correspondence Address: LEIGH HUNT FIRESTONE, 7100 NORFOLK ROAD, BERKELEY, CA, 94705, US

(73) Assignee: LEIGH HUNT FIRESTONE, BERKELEY, CA (US)

(21) Appl. No.: 11/746,525

(22) Filed: May 9, 2007

(51) Int. Cl. A61F 2/12

(52) U.S. Cl. 623/8

(57) ABSTRACT

The invention is articles of layered sheets or balled strips or pieces of extracellular matrix for forming breast implants for augmenting or reconstructing breast tissue in humans. The invention is also to methods of using these in implant articles to augment or reconstruct a human breast.

### CROSS-REFERENCES TO RELATED APPLICATIONS

The present application is not related to any other applications.

### FIELD OF THE INVENTION

The invention relates to augmenting or reconstructing a human breast using novel articles of mammalian extracellular matrix material.

### BACKGROUND OF THE INVENTION

Tissue regeneration has been accomplished by using extracellular matrix material derived from mammalian tissues. Some of these mammalian tissues that have been described in patent literature include small intestine submucosa (SIS), liver basement membrane (LBM), urinary bladder submucosa (UBS) and stomach submucosa (SS). See U.S. Pat. No. 5,554,389, U.S. Pat. No. 4,902,508, and U.S. Pat. No. 5,281,422. Enamel matrices, which are the extracellular matrix around forming teeth, are described in U.S. Pat. No. 7,033,611. Extracellular matrices from these tissues have been isolated and dried to become solid materials (sheets and particulates). Particulate forms can be rehydrated in a suitable buffer to become fluidized or emulsion forms. Presently, these extracellular matrix compositions are used for tissue grafting, wound healing, and tissue regenerative purposes.

### SUMMARY OF THE INVENTION

The invention is an article for placing in a human breast comprising: a plurality of sheets of mammalian extracellular matrix forming an implant shaped to conform to a desired shape of a human breast.

The invention is also an article for placing in a human breast comprising: a plurality of strips of mammalian extracellular matrix interlaced and balled to form an implant shaped to conform to a desired shape of a human breast.

The invention is also a method of augmenting and reshaping a human breast comprising: a. surgically opening said human breast, b. providing an article comprising graduated sheets of extracellular matrix for placing in said human breast forming an implant shaped to conform to a predetermined shape and size of said human breast, or providing an article comprising a plurality of strips of mammalian extracellular matrix interlaced and balled to form an implant shaped to conform to a desired shape of said human breast, c. placing said article of sheets or said article of strips of extracellular matrix in said human breast, and d. dosing said breast.

### BRIEF DESCRIPTION OF THE DRAWINGS

FIG. 1A depicts an article of concentric sheets of extracellular matrix.

FIG. 1B depicts a side view of an article of concentric sheets of extracellular matrix.

FIG. 2A depicts a side view of an article of concentric sheets of extracellular matrix with a single thread attaching the sheets to each other.

FIG. 2B depicts a side view of an article of concentric sheets of extracellular matrix with a several threads attaching the sheets to each other.

FIG. 5 depicts sectional views of introduction of extracellular matrix material into human lips for augmentation purposes.

FIG. 3A depicts a human breast having an article of concentric sheets placed in it.

FIG. 3B depicts a human breast having an article of concentric sheets placed in it, but the sheets decrease in area from the middle out to the nipple and also from the middle to a base position in the breast.

FIG. 4A depicts a side view of a ball of strips and pieces of extracellular matrix balled and shaped to conform to the shape of a human breast.

FIG. 4B depicts a view looking down on a ball of strips and pieces of extracellular matrix balled and shaped to conform to the shape of a human breast.

### DETAILED DESCRIPTION OF THE INVENTION

The invention is an article made of extracellular matrix sheets layered on top of one another, generally in some form of concentric layering to form an article to place in a breast. The article is useful for placing in a breast of a mammal in need of tissue regeneration, or tissue augmentation, in order to effect tissue regeneration or augmentation at the breast site where the article is placed. The extracellular matrices used in the article can be from one or more than one source of extracellular matrix in a mammal. The article is made of solid pieces of extracellular matrix, generally sheets or strips.

The sheets, strips or pieces can be from the same source of extracellular matrix, i.e. made of SIS from a pig. The sheets, strips, or pieces can also be from different sources of extracellular matrix, for example the several sheets can be SIS, alternated with sheets of SS, or the strips and pieces can be from both SIS and SS. Likewise the SIS and SS can be from the same species of mammal (pig) or each from a different species of mammal (SIS from pig, and SS from cow).

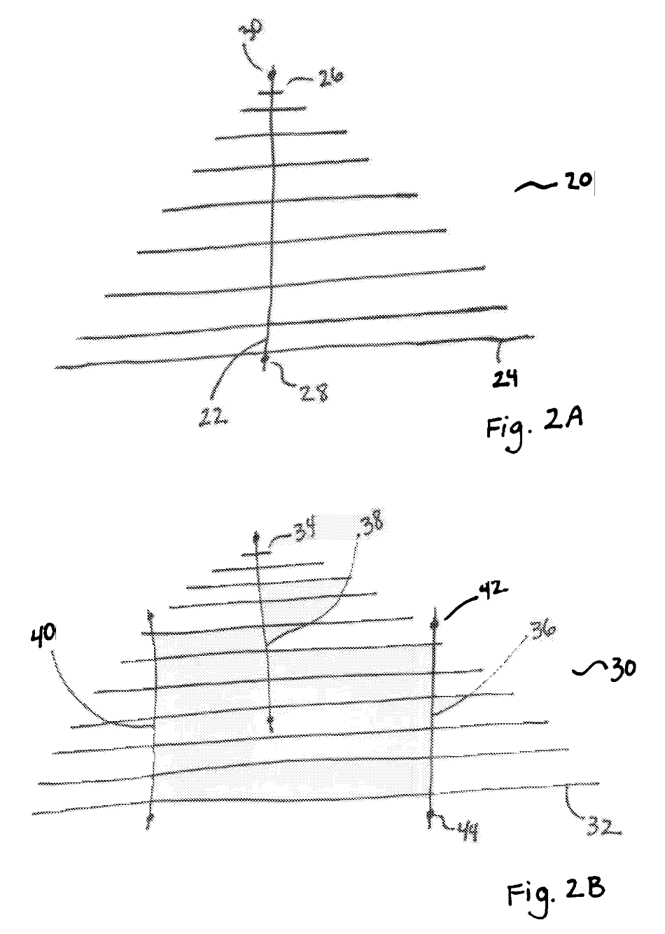

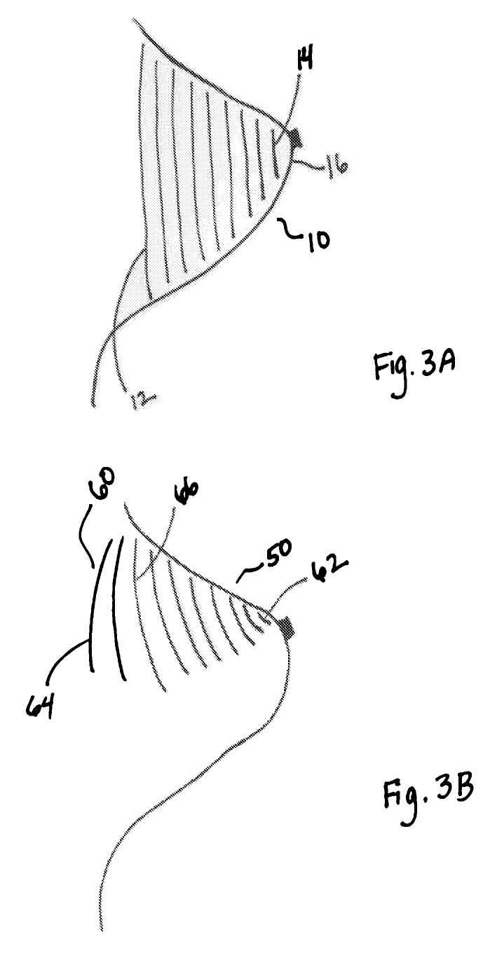

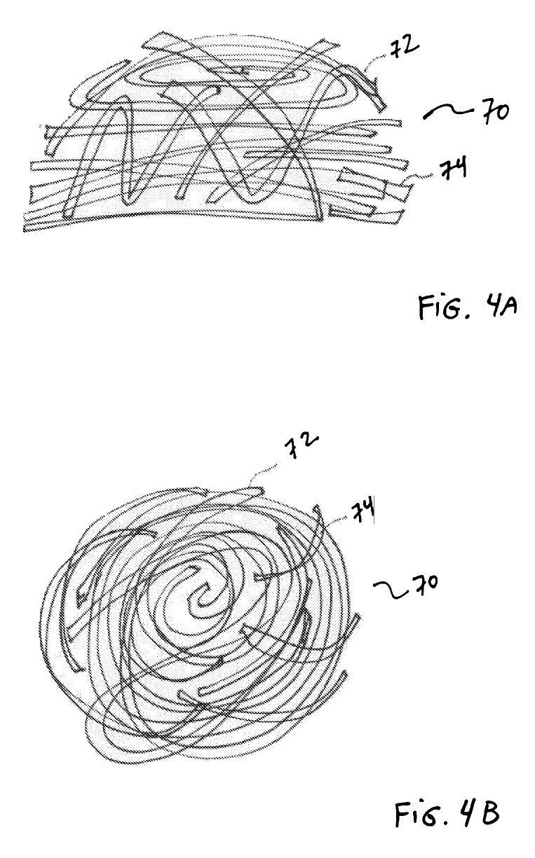

The sheets can be laminated to each other at the edges or they can be attached to one another by a thread or string or filament, or several threads or strings or filaments as depicted in FIGS. 2A and 2B. The filaments can be biodegradable material. Likewise a ball of pieces or strips can be loosely woven together together with one or more filaments of some biodegradable material, for example resolvable suture. The use of a filament to hold the sheets of extracellular matrix loosely together, or a ball of extracellular matrix strips and pieces loosely together in a shape consistent with a breast shape is done to ensure that the sheets or pieces or strips or extracellular matrix retain a proper form until and while being placed in the breast. Thereafter, the position of the article should be supported by the tissue regeneration process that ensues as the extracellular matrix becomes part of the breast tissue in the subject receiving the article.

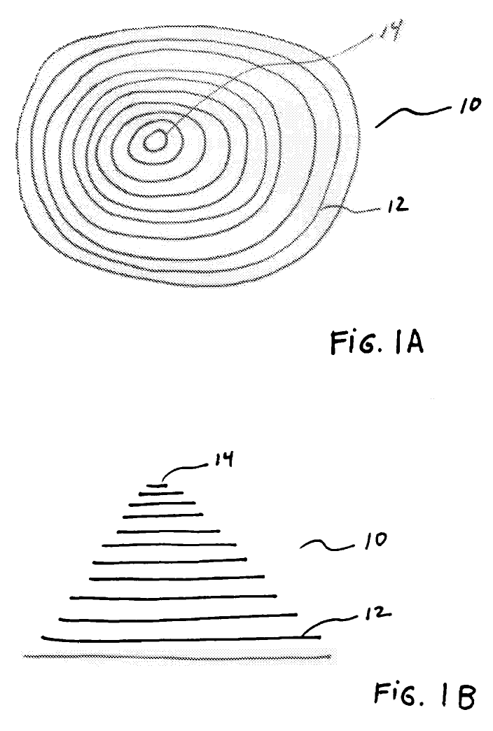

The sheets of extracellular matrix may also be attached to each other by quilting of the sheets in the middle much like a quilt is assembled when made of 2 or more layers of fabric. In order to form an article that is shaped like a human breast, several sheets may be laminated together to form the breast-like shape, for example concentric circles of extracellular matrix as depicted in FIG. 1A and FIG. 1B. The base of the article can be flat which conforms to the base of the human breast, and the top of the article will need to rise in a mound towards the nipple in order to conform to the natural or desired shape of the breast being augmented.

Mammalian tissue sources are in general any tissue having an extracellular matrix that can be isolated from a mammal and de-cellularized. Thus for example, most mammalian organs are tissue sources. The tissue sources can be for example any mammalian tissue, including but not limited to the small intestine, large intestine, stomach, lung, liver, kidney, pancreas, placenta, heart, bladder, prostate, tissue surrounding growing tooth enamel, tissue surrounding growing bone, and any fetal tissue from any mammalian organ.

The forms of the extracellular matrices that make up the articles are generally any form of extracellular matrix, including forms such as sheets, strips, or pieces, or other forms that result from human manipulation with the extracellular matrix. The solid form is generally a sheet of extracellular matrix. Sheets can be cut into strips or pieces for creating the balled article embodiment.

Extracellular matrix can be obtained from the tissues of mammals by processes such as described in U.S. Pat. No. 5,554,389, U.S. Pat. No. 4,902,508, and U.S. Pat. No. 5,281,422. For example, the urinary bladder submucosa is an extracellular matrix that has the tunica mucosa (which includes the transitional epithelial layer and the tunica propria), a submucosal layer, 3 layers of muscularis, and the adventitia (a loose connective tissue layer). This general configuration is true also for small intestine submucosa (SIS) and stomach submucosa (SS). Obtaining enamel matrices is described in U.S. Pat. No. 7,033,611. Enamel matrix is extracellular matrix existing near forming teeth.

Other tissues such as the liver and pancreas have a basement membrane that does not demonstrate the kind of tensile strength of the tissues defined as submucosa. However, other useful properties may be opportunistically employed from the extracellular matrices of such tissues as the liver, pancreas, placenta and lung tissues which have either basement membrane for extracellular matrix or interstitial membrane (as with the lung). These softer matrices support cells such as those in the organs from which the matrices are derived. Thus, certain benefits are to be found in using the extracellular matrices of these tissues, especially in combination with other such matrices like SIS and SS that may be stronger and which offer their particular advantages. The extracellular matrices surrounding developing tooth enamel and developing bone also have particular advantages over other matrices in that they support the growth and differentiation of the hard tissues of bone and enamel. Accordingly, the liver, lung, and pancreatic extracellular matrices may be quite suitable for generating some of the sheets, strips or pieces of the articles of the invention, and may be used as such.

The article comprising sheets, strips, or pieces of extracellular matrix can comprise extracellular matrix combinations from such sources as, for example but not limited to, small intestine submucosa, liver basement membrane, stomach submucosa, urinary bladder submucosa, placental basement membrane, pancreatic basement membrane, large intestine submucosa, lung interstitial membrane, respiratory tract submucosa, heart extracellular matrix, dermal matrix, and in general extracellular matrix from any mammalian fetal tissue. Any one of these tissue sources can provide extracellular matrix that can then be manipulated into a designated form (e.g. sheet, strip or piece) for use in the articles of the invention.

The articles of the invention that are made of sheets, strips, or pieces of extracellular matrix can be made from a single source of extracellular matrix. The composition can also be made from two or more extracellular matrices isolated from a donor mammal or from a particular tissue source in that donor or multiple donors. In any event, the key factor is that at least two tissue sources from which the composition comprising mammalian extracellular matrix can be derived to form the composition derived from different tissue sources.

The articles can be made from three mammalian tissue sources, four mammalian tissue sources, 5 mammalian tissue sources, 6 mammalian tissue sources, and conceivably up to 10 or more tissue sources. Once again these tissue sources can be from the same mammal (for example the same cow, the same pig, the same rodent, the same human, etc.), the same species of mammal (e.g. a cow, a pig, a rodent, a human), or different mammalian animals (but the same species, e.g. cow 1 and cow 2, or pig 1 and pig 2), or different species of mammals (for example liver matrix from a pig, and small intestine submucosa from a cow, and urinary bladder submucosa from a dog).

Accordingly, the articles can be made entirely for example of small intestine submucosa (SIS).

Turning now to the Figures, FIG. 1A depicts a downward looking view of a breast implant 10 having concentric sheets of extracellular matrix such as sheet 12 graduated down to sheet 14 which would rest just under the nipple. FIG. 1B depicts a sideview of breast implant article 10, having sheet 12 near the base, and sheet 14 at the tip of the implant.

FIG. 2A depicts a sideview of article 20 having concentric sheets connected by a bioabsorbable filament 22 connecting sheets 24 at the base to sheet 26 at the tip. Knots 28 and 30 secure the filament to the article. FIG. 2B depicts a second side view of an article 30 having concentric sheets having sheet 32 at the base and sheet 34 at the tip. Bioabsorbable filaments 36, 38 and 40 hold the concentric sheets together, and each have knots such as depicted for string 36 at points 42 and 44.

FIG. 3A depicts an implant 10 placed in a human breast. Sheet 12 is near the based and sheet 14 is at the tip. Breast 16 is pleasantly shaped and augmented by placing article 10 in the breast. FIG. 3B depicts article 50 placed inside a human breast. Due to the needs of the subject, the article for this subject required implant 60 which has a largest sheet 66 in the middle of the implant, and graduated layers to the tip 62 from the center, and further (mirror-like) graduated layers to the base of the breast resolving in small sheet 64. Implant 60 is to demonstrate that for the purposes of breast augmentation, sometimes partial material to fill part of the breast will suffice, as in reconstruction of a sagging breast or a breast otherwise diminished after years of breast feeding.

FIG. 4A depicts a side view of the balled article 70 of strips 72 and pieces 74 of extracellular matrix. FIG. 4B depicts a down-ward view of article 70 having strips 72 and pieces 74.

The invention is a method of augmenting and reshaping a human breast by surgically opening the breast and placing either the implant of concentric sheets, or the implant with the balled strips and pieces of extracellular matrix in the patient. The opening can be a typical opening used in the practice of plastic surgery involving breast augmentation or reconstruction. Generally the surgeon determines what size opening is necessary to place into the breast the implant. The articles used for the implants are made exclusively of extracellular matrix material in sheets (concentric sheets as depicted in FIGS. 1A and 1B), or balled strips and pieces as depicted in FIGS. 4A and 4B. The breast is then closed after placement of the article in the breast. In addition, before closure, the area can be sprinkled with dry extracellular matrix powder to facilitate tissue regeneration in the breast over and around the article. The particulate extracellular matrix is made from lyophilizing a sheet of extracellular matrix and crushing it into a powder form. The particulate can be made from any mammalian extracellular matrix, for example SIS, SS, LBM, UBS, and in general extracellular matrix from any tissue source.

The invention contemplates using the articles of extracellular matrices for placing in a human breast to facilitate augmentation or reconstruction of the breast. The specific article for a particular subject can be designed according to the needs of the breast size and shape. Accordingly, a female requiring a size D cup breast will have a larger implant than a female requiring a size C cup breast. Given the basic parameters of the construction of the articles for layered sheets or balled strips and pieces of extracellular matrix these size adjustments can be made in the product and even specially made for a particular recipient. The advantages of using extracellular matrix as a material for breast augmentation or reconstruction (after e.g. mastectomy, or partial mastectomy) is that unlike plastics that presently comprise breast implants of saline or silicone, extracellular matrix will incorporate into the breast and become breast tissue, relieving the physiological burden on the recipient of having a foreign article in her body. In addition, sometimes plastic implants need to be replaced which would not be the case with an implant of extracellular matrix that eventually incorporates itself into the breast tissue. In addition, scarring generally occurs with the placement of a plastic implant in the breast, leading to a hardening of the breast tissue around the implant. With the herein described implants of extracellular matrix, scarring does not occur, because extracellular matrix promotes scar-free tissue regeneration.

Regenerating tissue is the ability to make tissue regrow, an organ regrow itself, and for new tissue to reform without scarring, all of which are useful concepts for reconstructing breast tissue after a mastectomy or other tissue loss procedure. Healing a wound is the ability of the tissue to heal without scarring, or with less scarring than would have occurred without the article. Augmenting tissue is providing new material from which new tissue can form within the body.

All references cited are incorporated in their entirety. Although the foregoing invention has been described in detail for purposes of clarity of understanding, it will be obvious that certain modifications may be practiced within the scope of the appended claims.

What is claimed is:

1. An article for placing in a human breast comprising:
a plurality of sheets of mammalian extracellular matrix forming an implant shaped to conform to a desired shape of a human breast.

2. The article of claim 1, wherein said sheets are layered but otherwise unattached to each other and can be placed in said human breast individually from largest to smallest to achieve a desired shape and augmentation of said human breast.

3. The article of claim 1, wherein said sheets are attached to each other by one or more biodegradable filaments placed through said sheets attaching one or more of said sheets to another sheet.

4. The article of claim 1, wherein said sheets are spot laminated to one another in succession to form an integral unit of layers of extracellular matrix sheets.

5. The article of claim 1, wherein said sheets of mammalian extracellular matrix comprise small intestine submucosa.

6. The article of claim 1, wherein said sheets of mammalian extracellular matrix comprise stomach submucosa.

7. The article of claim 1, wherein said sheets of mammalian extracellular matrix comprise liver basement membrane.

8. The article of claim 1, wherein said sheets of mammalian extracellular matrix comprise urinary bladder submucosa.

9. An article for placing in a human breast comprising:
a plurality of strips of mammalian extracellular matrix interlaced and balled to form an implant shaped to conform to a desired shape of a human breast.

10. The article of claim 9, wherein said strips of mammalian extracellular matrix comprise small intestine submucosa.

11. The article of claim 9, wherein said strips of mammalian extracellular matrix comprise stomach submucosa.

12. The article of claim 9, wherein said strips of mammalian extracellular matrix comprise liver basement membrane.

13. The article of claim 9, wherein said strips of mammalian extracellular matrix comprise urinary bladder submucosa.

14. A method of augmenting or reconstructing a human breast comprising:
a. surgically opening said human breast,
b. providing an article comprising graduated sheets of extracellular matrix for placing in said human breast forming an implant shaped to conform to a predetermined shape and size of said human breast, or providing an article comprising a plurality of strips of mammalian extracellular matrix interlaced and balled to form an implant shaped to conform to a desired shape of said human breast.
c. placing said article of sheets or said article of strips of extracellular matrix in said human breast,
d. closing said breast.

15. The method of claim 14, further comprising dusting said article after placement in said breast with extracellular matrix particulate before closure of said breast.
